(19) United States
(12) Patent Application Publication
(10) Pub. No.: US 2006/0104931 A1
(43) Pub. Date: May 18, 2006

(54) Cosmetic treatment article comprising substrate and gel composition

(76) Inventors: Takeshi Fukutome, Ohtsu (JP); Miwa Miyamoto, Kobe (JP); Kazuyuki Ohnishi, Takaishi (JP); David Leigh Trigg, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/268,858

(22) Filed: Nov. 8, 2005

Related U.S. Application Data
(60) Provisional application No. 60/627,698, filed on Nov. 12, 2004.

(51) Int. Cl. A61K 8/73; A61K 36/02

(52) U.S. Cl. 424/70.13; 424/195.17

(57) ABSTRACT

Disclosed are cosmetic treatment articles comprising: (1) a water-insoluble substrate; (2) a gel composition comprising: (a) a first gel forming composition comprising a water soluble polymeric gelling agent and (b) a second gel forming composition comprising a gel strengthening agent; and (3) preferably, a treatment composition comprising a rheology modifier. Further disclosed are cosmetic treatment articles comprising at least two different treatment compositions, each treatment composition being provided at one or more selected locations on the water insoluble substrate.

### CROSS REFERENCE TO RELATED APPLICATION

This application claims the benefit of U.S. Provisional Application No. 60/627,698, filed on Nov. 12, 2004.

### FIELD OF THE INVENTION

The present invention relates to a cosmetic treatment article for topical application, in particular a mask composition, wherein the cosmetic treatment article comprises a water insoluble substrate and a gel composition, and preferably also comprises a treatment composition.

### BACKGROUND OF THE INVENTION

Cosmetic treatment articles including masks designed for providing treatment to the skin are known in the art, such as SKII Facial Treatment Mask on the Japanese market. Such masks are made of a substrate and a liquid soaked in the substrate, wherein the mask is adhered only very weakly to the skin, such that the mask is easily removed from the skin with practically no tension to the skin. These treatment masks can be distinguished from removal masks. Removal masks are those designed to firmly adhere to the skin and thereby remove dirt, clogs, and excess corneum on the surface and in the pores of skin upon peeling off the mask. Treatment masks are particularly suitable for applying to the skin for delivering moisturizing agents and other benefit agents to the skin through a wet, typically aqueous, environment. In fact, delivery of moisturizing agents and other benefit agents via a mask is advantageous in that the skin is exposed to an abundant amount of such agents over a lengthy period of time. Treatment masks also provide relaxation benefit to the user upon use, because the usage encourages the user to sit or lie down. Treatment masks are generally applied to the facial skin.

Another type of mask that is commercially available for use on the facial skin is a hydrogel-based substrate mask, for example the “Lifecella” mask that is available in Japan from the Hisamitsu Company. This type of mask is easy to use, and offers good adhesion and fit to the skin, but is generally not as efficacious in delivering skin care actives.

Based on the foregoing, there is still a need for a cosmetic treatment article which provides improved skin care benefits, and particularly, a mask composition which provides improved use experience and adhesion to the skin while providing good delivery of skin benefit agents or actives.

None of the existing art provides all of the advantages and benefits of the present invention.

### SUMMARY OF THE INVENTION

The present invention is directed to a cosmetic treatment article comprising: (1) a water-insoluble substrate; (2) a gel composition comprising: (a) a first gel forming composition comprising a water soluble polymeric gelling agent and (b) a second gel forming composition comprising a gel strengthening agent; and (3) preferably, a treatment composition comprising a rheology modifier. The present invention is further directed to a cosmetic treatment article comprising: (1) a water insoluble substrate; (2) a gel composition comprising: (a) a first gel forming composition comprising a water soluble polymeric gelling agent; and (b) a second gel forming composition comprising a gel strengthening agent; and (3) at least two different treatment compositions, at least one of the treatment compositions comprising a rheology modifier, and each treatment composition being provided at one or more selected locations on the water insoluble substrate. The present invention is still further directed to a cosmetic treatment article kit comprising: (1) a water insoluble substrate; (2) a gel composition comprising: (a) a first gel forming composition comprising a water soluble polymeric gelling agent; (b) a second gel forming composition comprising a gel strengthening agent; and (3) a treatment composition comprising a rheology modifier; wherein the water insoluble substrate is maintained separate from at least one of the first gel forming composition, the second gel forming composition, and the treatment composition until the time of use of the article.

These articles can provide skin care benefits such as hydration to the skin upon use, while providing good fit and adhesion to the skin. They are also easy and convenient to use.

These and other features, aspects, and advantages of the present invention will become better understood from a reading of the following description, and appended claims.

### BRIEF DESCRIPTION OF THE DRAWINGS

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is a plan view of a preferred embodiment of the water-insoluble substrate of the present invention.

### DETAILED DESCRIPTION OF THE INVENTION

While the specification concludes with claims particularly pointing out and distinctly claiming the invention, it is believed that the present invention will be better understood from the following description.

While the article of the present invention will be described in the context of a facial mask, one of ordinary skill in the art will recognize the ability to and be able to readily adapt the present invention to cosmetic articles in general. As used herein, the term “cosmetic article” refers to devices which are adapted for application to the body, and in particular, to the human body. More specifically, “cosmetic articles” are devices for personal care or cosmetic devices, including wipes, facial masks and the like.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “topical application” means to apply or spread a material onto the surface of the skin.

Herein, “cosmetically-acceptable” means that the compositions or components thereof so described are suitable for use in contact with human skin without undue toxicity, incompatibility, instability, allergic response, and the like.

Herein, “mixtures” is meant to include a simple combination of materials and any compounds that may result from their combination.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Water-Insoluble Substrate

A preferred embodiment of the present invention comprises a cosmetic treatment article such as a treatment mask for application to the human body. The article comprises a water-insoluble substrate. By “water insoluble”, it is meant that the substrate does not dissolve in or readily break apart upon immersion in water. The water-insoluble substrate is the implement or vehicle for delivering the treatment composition to the skin.

A wide variety of materials can be used as the substrate. The following nonlimiting characteristics are desirable: (i) sufficient wet strength for use, (ii) sufficient softness, (iii) sufficient thickness, (iv) appropriate size, (v) air permeability, and (vi) hydrophilicity.

Nonlimiting examples of suitable substrates which meet the above criteria include nonwoven substrates, woven substrates, hydroentangled substrates, air entangled substrates, natural sponges, synthetic sponges, polymeric netted meshes, and the like. Preferred embodiments employ nonwoven substrates since they are economical and readily available in a variety of materials. By “nonwoven”, it is meant that the layer is comprised of fibers which are not woven into a fabric but rather are formed into a sheet, mat, or pad layer.

The substrates may be comprised of a variety of materials both natural and synthetic. Nonlimiting examples of natural materials useful in the present invention include: silk fibers; keratin fibers such as wool fibers and camel hair fibers; and cellulose fibers such as wood pulp fibers, cotton fibers, hemp fibers, jute fibers, and flax fibers. Nonlimiting examples of synthetic materials useful in the present invention include: acetate fibers; acrylic fibers; cellulose ester fibers; polyamide fibers; polyester fibers such as polyethylene terephthalate fibers; polyolefin fibers such as polypropylene fibers and polyethylene fibers; polyvinyl alcohol fibers; rayon fibers; and polyurethane foam.

Substrates useful in the present invention can also be obtained from a wide variety of commercial sources. Nonlimiting examples of suitable nonwoven substrates useful herein include the following. Havix 2360 is a single-layer substrate (carded and air-through nonwoven) having a layer of PE/PP bicomponent fiber (90%) and PE/PET bicomponent fiber (10%). This Havix nonwoven is chemically treated to be more hydrophilic. The total basis weight is about 23 gsm. Further suitable is Kuraray TT463Q60, a single-layer substrate (carded & hydro-entangled nonwoven) having a layer of PET fiber. The PET resin is chemically treated to be more hydrophilic. The total basis weight is about 60 gsm. Also suitable are WALKISOFT®, a cellulose substrate available from Walkisoft U.S.A.; NOVONET® 149-801 and 149-191, a substrate containing about 69% rayon, about 25% polypropylene, and about 6% cotton, available from Veratec, Inc. Walpole, Mass.; KEYBAK® 951V and 1368, a substrate containing about 75% rayon and about 25% acrylic fibers, available from PGI/Chicopee, Dayton, N.J.; RMT-90, a 3-layer substrate having a pulp layer as an inner layer with outer layers respectively made of the combination of rayon and polyester, and RFP-90, a 3-layer substrate having 30 gsm PP carded & thermal bonded nonwoven as an inner layer and 60 gsm rayon fiber as outer layers (each side has 30 gsm). The outer layers were webbed using carding process and they are combined with the inner layer using hydro-entangled process. RMT-90 and RFP-90 are available from Daiwabo K.K.

The substrate can be made into a wide variety of shapes and forms such as flat pads, thick pads, thin sheets, and sheets of irregular thickness, depending on the desired use and characteristic of the mask. The substrate is typically designed to fit the area of the skin to which topical application is desired. For example, when the mask is applied to the face, the substrate is designed to correspond to the shape of the face avoiding the eye, nostril, and mouth areas, as necessary.

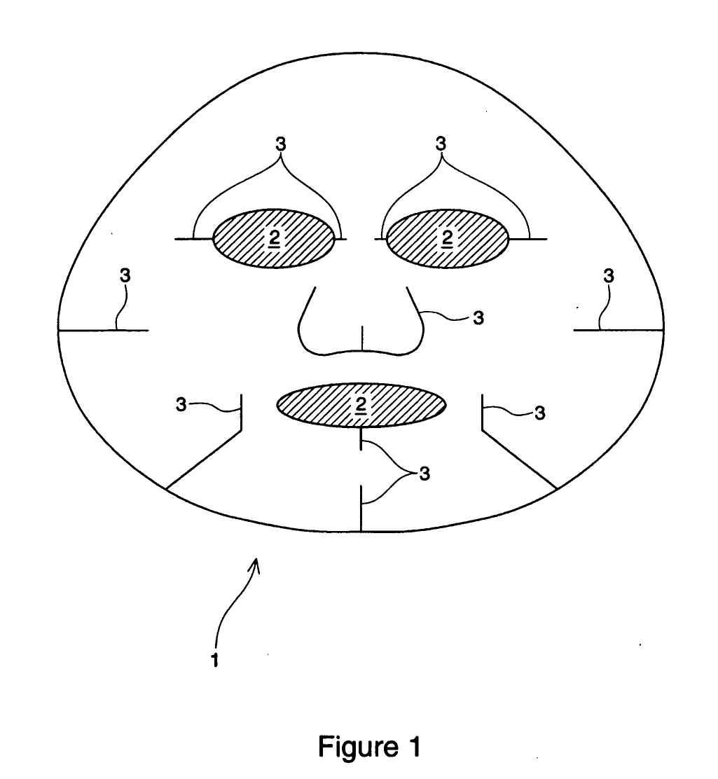

In one preferred embodiment, the substrate is so configured to cover substantially the whole area of the facial skin with areas of the eyes and nostrils opened. Referring to FIG. 1, a plane view of a particularly preferred embodiment of a substrate suitable for a single-piece whole facial mask (10) is depicted. The outer peripheral of the substrate of FIG. 1 is designed to approximately match the contour of the face, with a plurality of openings (12) for the eyes and the mouth, and wherein a plurality of cuttings (13) are made so that the mask fits the nose, cheeks, and the mouth. The embodiment of FIG. 1 has a length of from about 15 cm to about 25 cm, preferably from about 18 cm to about 23 cm, and a width of from about 15 cm to about 30 cm, preferably from about 20 cm to about 25 cm; to cover the average entire facial area. In another preferred embodiment, the substrate is so configured to cover substantially the whole area of the facial skin, and is made of two pieces, the first piece covering the upper area of the face, i.e. the nose and thereabove, and the second piece covering the lower area of the face, i.e. the upper lip, cheeks and thereunder. In another preferred embodiment, the substrate is so configured to match the area of a particular part of the face, such as the nose, cheekbone, chin, forehead, or combinations thereof. In another preferred embodiment, the substrate is so configured to have ears, pulls, or rings for facilitating placement and/or removal of the mask on the skin.

The substrate is flexible enough such that, when impregnated with the gel composition, it readily fits along the skin, yet is strong enough so that it does not easily tear or crumble upon use. Preferably, the substrate has a thickness of from about 100 μm to about 1 cm, more preferably from about 300 μm to about 3 mm, depending on the material for making the substrate, and use and characteristic of the product.

Substrate materials particularly useful herein include those which are of hydrophilic nature, thereby capable of absorbing a larger quantity of the gel composition. The water-insoluble substrate can be made solely of hydrophilic material, or made of a mixture of hydrophilic material and hydrophobic material. The substrates of the present invention can consist of a single layer or multiple layers. In one preferred embodiment, the substrate is made of at least partially by hydrophilic materials selected from cotton, pulp, rayon, and mixtures thereof. By partially, it is meant to encompass following situations: where one layer of a hydrophilic material is used for a single layered substrate; where at least one layer of a hydrophilic material is used for a multiple layered substrate; where one layer of a mixture of the hydrophilic material and another material is used for a single layered substrate; and where at least one layer of a mixture of the hydrophilic material and another material is used for a multiple layered substrate.

When the substrate consists of multiple layers, it is preferred that at least the layer facing the skin is that of hydrophilic nature, thereby capable of absorbing a larger quantity of the gel composition. When the substrate consists of multiple layers, the substrates can include films and other nonfibrous materials. In one embodiment, the substrate may also be laminated with polymeric film on the substrate, coating the substrate, or heat sealing the substrate. The resulting substrate with the laminated polymeric film, coating or heat sealing comprises an occluded side on one side of the substrate, which faces away from the skin, and a skin facing side that is positioned on the skin surface. By having a substrate with an occluded side, the substrate acquires low air permeability. By “low air permeability” it is meant that the side of the substrate having the film, coating or heat sealing allows very little air to enter into the substrate and very little vapor to escape from the substrate. Preferably the air permeability is less than about 5 mg/cm²/min, more preferably between about 0.01 mg/cm²/min and about 4.8 mg/cm²/min. The air permeability can be measured by taking the weight of a fully saturated sample of the substrate and weighing the substrate after it is exposed to the atmosphere.

Gel Composition

The treatment articles of the present invention comprise a gel composition in addition to the water-insoluble substrate described hereinbefore. The gel composition is comprised of: (a) a first gel forming composition comprised of a water soluble polymeric gelling agent; and (b) a second gel forming composition comprised of a gel strengthening agent.

First Gel Forming Composition

The first gel forming composition is comprised of a water soluble polymeric gelling agent selected from synthetic or natural polymers, and mixtures thereof. Preferred are natural polymers, including gelatin, polysaccharides, and mixtures thereof. The polysaccharides for use herein include red seaweed polysaccharides; glucomannans; galactomannans; fermentation polysaccharides or derivatives thereof; brown seaweed polysaccarides; extracts of marine invertebrates; starch or derivatives thereof; natural fruit extracts, plant fiber derivatives; kelp; natural plant exudates and resinous gums; and mixtures thereof.

Brown seaweed polysaccharides are isolated by extraction for various species of Phaebophyceae. Suitable brown seaweed polysaccharides for use herein include algin, alginic acid and salts thereof, potassium alginate, calcium alginate, sodium alginate, and ammonium alginate, propylene glycol alginate, and mixtures thereof. Herein, sodium alginate and potassium alginate are preferred alginates.

Red seaweed polysaccharides are isolated from marine plant species belonging to the class of Phodophyceae. Red seaweed polysaccharides provide mechanicl strength in an aqueous gel. Suitable red seaweed polysaccharides for use herein include agar known in the industry under the CFTA trade designation agar agar flake derived from various Gelidium plant species or closely related red algae commercially available as Agar Agar 100 from Gumix International Inc. (Fort Lee N.J., USA); agarose commercially available as Sea Plaque from FMC (Philadelphia, Pa., USA) and Agarose Type 1-b from Sigma Aldrich Co. Ltd. (Poole, UK); carrageenan, comprising the fractions lambda-, iota- and kappa- which are the water extracts obtained from various members of the Gigartinaceae or Solieriaceae families, known in the industry under the (CTFA) trade designation as chondrus, commercially available as “Gelcarin® LA”, “Seakem® 3/LCM”, or “Viscarin® XLV”, all from FMC (Philadelphia, Pa., USA); and furcellaran commercially available from Gum Technology Corporation (Tucson, Ariz., USA) and Continental Colloids Inc. (Chicago, Ill., USA), or mixtures thereof. Preferably, the red seaweed polysaccharide for use herein is selected from agar, agarose, kappa-carrageenan and furcellaran, or mixtures thereof.

Glucomannans are polysaccharides which comprise an essentially linear backbone of glucose and mannose residues. Glucomannans have short side branches attached to the linear backbone and acetyl groups are randomly present at the C-6 position of a sugar unit. The acetyl groups are generally found on one per six sugar units to one per twenty sugar units. Suitable glucomannans or derivatives thereof for use herein have a ratio of mannose to glucose of from about 0.2 to about 3. Preferred glucomannans for use herein include konjac mannan, which is the generic name for the flour formed from grinding the tuber root of the Amorphophallus konjac plant (elephant yam), commercially available under the trade name “Nutricol® konjac flour” from FMC (Philadelphia, Pa., USA); and deacetylated konjac mannan; or mixtures thereof.

Galactomannans are vegetable reserve polysaccharides which occur in the endosperm cells of numerous seeds of Leguminosae. The collective term “galactomannan” comprises all polysaccharides which are built up of galactose and mannose residues. Galactomannans comprise a linear backbone of (1→4)-linked β-D-mannopyranosyl units. To these rings are attached as branches, isolated galactopyranose residues by α-(1,6)-glucoside bonds. Galactomannans may in addition also contain minor amounts of other sugar residues. Suitable galactomannans for use herein are fenugreek gum; lucern; clover; locust bean gum known for example in the industry under the (CTFA) trade designation as carob bean gum, commercially available as “Seagul L” from FMC (Philadelphia, Pa., USA); tara gum commercially available from Starlight Products (Rouen, France) or Bunge Foods (Atlanta, Ga., USA); guar gum derived from the ground endosperms of Cyamopsis tetragonolobus, commercially available as “Burtonite V7E” from TIC Gums (Belcamp, Md., USA), “Jaguar C” from Rhone-Poulenc (Marietta, Ga., USA), or “Supercol” from Aqualon (Wilmington, Del., USA); and cassia gum commercially available from Starlight Products (Rouen, France), or mixtures thereof. Preferably, the galactomannans for use herein have an average one of every 1 to about 5 mannosyl units substituted with a (1→6)-linked-α-D-galactopyranosyl unit and are selected from guar gum, locust bean gum and cassia gum, or mixtures thereof.

Fermentation polysaccharides are polysaccharides which are commercially produced by the fermentation of micro-organisms in a medium containing a carbon and nitrogen source, buffering agent, and trace elements. Suitable fermentation polysaccharides or derivatives thereof, for use in the present invention include gellan gum known in the industry under the (CTFA) trade designation as gum gellan, a high molecular weight hetero polysaccharide gum produced by a pure-culture fermentation of a carbohydrate with Pseudomonas elodea, commercially available as “Kelcogel” from Kelco (San Diego, Calif., USA); xanthan gum which is a high molecular weight hetero polysaccharide gum produced by a pure-culture fermentation of a carbohydrate with Xanthomonas campestris, known in the industry under the (CTFA) trade designation as xanthan, commercially available for example as “Keltrol CG 1000/BT/F/GM/RD/SF/T/TF”, from Calgon (Pittsburgh, Pa., USA), or “Kelzan” from Kelco (San Diego, Calif., USA); natto gum; pullulan; rhamsan gum; curdlan; succinoglycan; welan gum; dextran, commercially available as “Sephadex G-25” from Pharmacia Fine Chemicals (Piscataway, N.J., USA) and derivatives thereof; and sclerotium gum, commercially available as “Amigel” from Alban Muller International (Montreil, France), or mixtures thereof. Preferred fermentation polysaccharides or derivatives thereof are selected from gellan gum and xanthan gum, or mixtures thereof. More preferably the fermentation polysaccharide or derivative thereof is xanthan gum.

Extracts of marine invertebrates can also be used. Polysaccharides derived from marine invertebrates, specifically the exoskeleton of such invertebrates, consist chiefly of N-acetyl-D-glucosamine residues. Examples of such polysaccharides suitable for use herein include chitosan, commercially available for example as “Marine Dew” from Ajinomoto (Teakneck, N.J., USA); and hydroxypropyl chitosan commercially available for example as “HPCH Liquid” from Ichimaru Pharcos (Yamagata Gun Gifu-Pref, Japan) and derivatives; or mixtures thereof.

Starches are polysaccharides which consist of various proportions of two glucose polymers, amylose and amylopectin. Suitable materials for use herein include starch (for example, rice starch, corn starch, potato starch, wheat starch, and mixtures thereof), amylopectin and dextrin, commercially available as “Nadex 360” from National Starch (Bridgewater, N.J., USA), and derivatives or mixtures thereof. Examples of natural fruit extracts suitable for use herein include pectin and salts thereof such as sodium pectate, arabian and mixtures thereof. A suitable example of a plant fiber derivative for use herein is cellulose. Suitable polysaccharides obtained from natural plant exudates for use herein include karaya, tragacanth, arabic, tamarind, and ghatty gums, or mixtures thereof. Examples of resinous gums suitable for use herein include shellac gum, which is obtained from the resinous secretion of the insect Laccifer (Tachardia) lacca, damar gum; copal gum and rosin gum; or mixtures thereof.

The water soluble polymeric gelling agent is included in the first gel forming composition at a level by weight of, preferably from about 0.01% to about 10%, more preferably from about 0.05% to about more preferably from about 0.05% to about 5%, still more preferably from about 0.1% to about 2%.

The first gel forming composition desirably further comprises a carrier such as water and/or glycerin, and also may include preservatives and preservative enhancers such as water-soluble or dispersible preservatives including methyl paraben, ethyl paraben, propyl paraben, imidazolidinyl urea, Germall 115, methyl, ethyl, propyl and butyl esters of hydroxybenzoic acid, benzyl alcohol, EDTA, Bronopol (2-bromo-2-nitropropane-1,3-diol), sodium salicyclate, phenoxypropanol, ethyl alcohol, iso-propyl alcohol, butylene glycol, pentylene glycol, hexylene glycol, and mixtures thereof. Other preservatives and preservative enhancers that are commonly used in cosmetic/skincare and quasi drug products may further be suitable for use herein.

Second Gel Forming Composition

The second gel forming composition is comprised of a gel strengthening agent, for example sugar, alcohol, any monovalent salt or multivalent salt or metal ion, and mixtures thereof. However, if alginates are used as the water soluble polymeric gelling agent of the first gel forming composition, magnesium and mercury metal ions are not suitable, as they are not compatible with alginates. Suitable cations for such salts can be selected from potassium, sodium, ammonium, zinc, aluminum, calcium, iron, and magnesium ions, and mixtures thereof. Suitable anions associated with the aforementioned cations may be selected from chloride, citrates, sulphate, carbonate, borate, and phosphate anions, and mixtures thereof. The gel strengthening agent is included in the second gel forming composition at a level by weight of, preferably from about 0.01% to about 10%, more preferably from about 0.05% to about more preferably from about 0.05% to about 5%, still more preferably from about 0.1% to about 2%.

The second gel forming composition desirably further comprises a carrier such as water, and also may include preservatives and preservative enhancers such as water-soluble or dispersible preservatives including methyl paraben, ethyl paraben, propyl paraben, imidazolidinyl urea, Germall 115, methyl, ethyl, propyl and butyl esters of hydroxybenzoic acid, benzyl alcohol, EDTA, Bronopol (2-bromo-2-nitropropane-1,3-diol), sodium salicyclate, phenoxypropanol, ethyl alcohol, iso-propyl alcohol, butylene glycol, pentylene glycol, hexylene glycol, and mixtures thereof. Other preservatives and preservative enhancers that are commonly used in cosmetic/skincare and quasi drug products may further be suitable for use herein.

When combined, the first gel forming composition and the second gel form a gel structure. Without being bound by theory, it is believed that ion exchange reaction occurs between the water soluble polymeric gelling agent of the first gel forming composition and the gel strengthening agent of the second gel forming composition. For example, when alginate which contains negatively charged carboxyl groups is used as a water soluble polymeric gelling agent and salts of divalent metals are used as a gel strengthening agent, the alginate chains become crosslinked via ionic bonding with the divalent cations that act as bridges. This change in chemical structure is accompanied by an increase in viscosity and the development of shorter flow characteristics. As the reaction proceeds, the alginate becomes further crosslinked and loses the ability to flow, until finally it gels.

When applied to the water insoluble substrate, such gel structure permeates the substrate, giving it a different texture, feeling, and fit characteristic as compared to the substrate per se. For this reason, hydrophilic substrates are preferred herein.

Treatment Composition

The cosmetic treatment articles of the present invention, in addition to the gel composition, preferably further include a treatment composition that provides at least one skin benefit agent. The treatment composition includes a rheology modifier. Suitable rheology modifiers include synthetic thickeners such as laponite and natural clays such as bentonite and hectorite. Herein, laponite is preferred due to its relatively smaller particle size and its ability to form a transparent gel-like treatment composition. An exemplary laponite suitable for use herein is XLG which can be obtained from Rockwood Additives, Ltd., of the UK. The rheology modifier is included in the treatment composition at a level by weight of, preferably from about 0.01% to about 20%, more preferably from about 0.1% to about 10%, still more preferably from about 1% to about 5%.

The presence of the rheology modifier provides the treatment compositions herein with different, preferably higher, viscosity characteristics as compared to liquid treatment compositions that have previously been provided with water-insoluble substrate facial masks. After application to the skin, the treatment compositions of the present invention increase in viscosity due to presence of the rheology modifier. The degree of viscosity increase depends on the amount of rheology modifier added to the treatment composition.

The treatment composition may further comprise an additional rheology modifier. An additional rheology modifier may help to improve the stability and physical properties of the treatment composition, and may help to prevent syneresis. The additional rheology modifiers suitable herein include water soluble or water miscible polymers that have the ability to increase the viscosity of the composition, and are compatible with other components used in the composition. The additional rheology modifier may be included, by weight of the treatment composition, at a level preferably from about 0.1% to about 5%, more preferably from about 0.1% to about 3%, still preferably from about 0.2% to about 2%.

Water soluble thickening polymers useful herein as the additional rheology modifier include anionic polymers and nonionic polymers. The water soluble thickening polymers useful herein include, for example, acrylic polymers, polyalkylene glycol polymers having a molecular weight of more than about 10000, celluloses and derivatives there of such as hydroxyethyl cellulose, polyvinylpyrrolidone, polyvinyl alcohol, gums such as guar gum and xanthan gum, carragenan, pectin, agar, quince seed (Cydonia oblonga Mill), starch (rice, corn, potato, wheat), algae colloids (algae extract), dextran, succinoglucan, pulleran, carboxymethyl starch, methylhydroxypropyl starch, sodium alginate, and alginic acid propylene glycol esters. Neutralizing agents may be included to neutralize the anionic thickening agents described hereinabove. Nonlimiting examples of such neutralizing agents include sodium hydroxide, potssium hydroxide, ammonium hydroxide, monethanolaamine, diethanolamine, triethanolamine, diisopropanolamine, aminomethylpropanol, tromethamine, tetrahydroxypropyl ethylenediamine, and mixtures thereof.

Among the above polymers, highly preferred are those providing reduced undesirable polymer flakes when treatment compositions are dried on the skin. Such highly preferred polymers include, for example, acrylic polymers. Acrylic polymers useful herein include those comprising monomers selected from the group consisting of acrylic acid, salts of acrylic acid, derivatives of acrylic acid, methacrylic acid, salts of methacrylic acid, derivatives of methacrylic acid, and mixtures thereof. The derivatives include, for example, alkyl acrylate, acrylamide, alkyl metahcrylate, and methacrylamide. Such acrylic polymers include, for example, cross linked acrylic acid polymers with the CTFA name Carbomer, sodium polyacrylate, polyethylacrylate, polyacrylamide, and acrylic acid/alkyl acrylate copolymers with the CTFA name Acrylates/C10-30 Alkyl Acrylate Crosspolymer. Commercially available acrylic polymers highly useful herein include, for example, polyacrylamide with tradename Sepigel 305 available SEPPIC Inc., and Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

The treatment composition of the present invention preferably includes at least one skin benefit agent. The skin benefit agent of the present invention includes those as known in the art and includes water soluble humectants, chronic whitening agents, skin tone changing agents or depigmentation agents, peptides, flavonoids, and mixtures thereof. The ingredients are described in more detail herein.

In addition, the skin benefit agent may comprise anti-oxidants, cleansing agents, free radical scavengers, moisturizers, skin tone altering agents, anti-acne agents, anti-dandruff agents, anti-aging agents, softeners, anti-wrinkle agents, keratolic agents, anti-inflamatory agents, skin texture treatment agents, fresheners, healing agents, liporegulators, vascular protectors, anti-bacterials, agents, anti-fungal agents, anti-perspirant agents, deodorants, skin conditioners, anesthetics, nourishing agents, sebum absorbers, and moisture absorbers. Such ingredients are generally included in the treatment composition at a level of typically no more than about 5% by weight of the treatment composition. The ingredients are described in more detail herein.

The treatment composition of the present invention preferably contains a water soluble humectant as a skin benefit agent. Water soluble humectants are preferably included to provide moisturizing benefit to the skin. Further, water soluble humectants may help the dispersion of the water soluble thickening agents, and dissolving/dispersion of other components which are relatively difficult to process in an aqueous carrier. The water soluble humectants may be included, by weight of the liquid composition, at a level preferably from about 0.1% to about 30%, more preferably from about 1% to about 20%, still preferably from about 5% to about 15%.

Water soluble humectants useful herein include polyhydric alcohols such as glycerin, diglycerin, propylene glycol, dipropylene glycol, butylene glycol, hexylene glycol, sorbitol, ethoxylated glucose, 1,2-hexane diol, hexanetriol, erythritol, trehalose, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein also include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

The treatment composition may include a chronic whitening agent as a skin benefit agent. The chronic whitening agent useful herein refers to active ingredients that not only alter the appearance of the skin, but further improve hyperpigmentation as compared to pre-treatment. By definition, chronic is referred to continued topical application of the composition over an extended period during the subject's lifetime, preferably for a period of at least about one week, more preferably for a period of at least about one month, even more preferably for at least about three months, even more preferably for at least about one year. Typically, applications would be on the order of about once per day over such extended periods, while application rates can vary from about once per week up to about three times per day or more. The chronic whitening agents may be included, by weight of the liquid composition, at a level preferably from about 0.001% to about 10%, more preferably from about 0.1% to about 5%.

Useful chronic whitening agents useful herein include ascorbic acid compounds, vitamin B₃ compounds, azelaic acid, butyl hydroxy anisole, gallic acid and its derivatives, glycyrrhizinic acid, hydroquinoine, kojic acid, arbutin, mulberry extract, ergothioneine, and mixtures thereof. Among them, preferred are ascorbic acid compounds, vitamin B₃ compounds, and mixtures thereof. Use of combinations of chronic whitening agents are believed to be advantageous in that they may provide whitening benefit through different mechanisms.

Ascorbic acid compounds useful herein include ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein includes, for example, esters of ascorbic acid, and ester salts of ascorbic acid. Particularly preferred ascorbic acid compounds include 2-o-α-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phospate ester salts such as sodium ascorbyl phophate, potassium ascorbyl phosphate, magnesium ascorbyl phosphate, and calcium ascorbyl phosphate. Commercially available ascorbic compounds include 2-o-α-D-glucopyranosyl-L-ascorbic acid available from Hayashibara and sodium L-ascorbyl phosphate with tradename STAY C available from Roche.

Vitamin B₃ compounds useful herein include, for example, those having the formula: 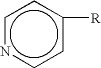
wherein R is —CONH₂ (e.g., niacinamide) or —CH₂OH (e.g., nicotinyl alcohol); derivatives thereof; and salts of any of the foregoing.

Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form.

The treatment compositions of the present invention may include a skin tone changing agent as a skin benefit agent. The skin tone changing agents useful herein are selected from the group consisting of skin tone changing pigments, reflective particulate material, and mixtures thereof. Skin tone changing agents useful herein are those altering the appearance of the color and/or tone of the skin including, but not limited to, skin whitening. The skin tone changing agents have a particle size of, preferably at least about 100 nm. The skin tone changing pigments useful herein include, for example, talc, mica, silica, magnesium silicate, titanium oxide, zinc oxide, and titanium oxide coated mica.

The reflective particulate materials useful herein have a primary particle size of from about 100 nm to about 10 μm (i.e., in the essentially pure, powder form prior to combination with any carrier). The reflective particulate materials can be inorganic. The inorganic reflective particulate materials useful herein include, for example, titanium dioxide, zinc oxide, more preferably the particles consist essentially of titanium dioxide. The inorganic reflective particulate materials can be coated with a coating material such as cationic polymers, cationic surfactants, anionic polymers, and anionic surfactants.

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

Suitable dipeptides for use herein include Carnosine® (beta-ala-his). Suitable tripeptides for use herein include, gly-his-lys, arg-lys-arg, his-gly-gly. Preferred tripeptides and derivatives thereof include palmitoyl-gly-his-lys, which may be purchased as Biopeptide CL® (100 ppm of palmitoyl-gly-his-lys commercially available from Sederma, France); Peptide CK (arg-lys-arg); PEPTIDE CK+ (ac-arg-lys-arg-NH₂); and a copper derivative of his-gly-gly sold commercially as IAMIN, from Sigma (St. Louis, Mo.). Tetrapeptides and pentapeptides are also suitable for use herein. A preferred commercially available pentapeptide derivative composition is palmitoyl-lys-thr-thr-lys-ser (commercially available from Sederma, France).

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition. In certain compositions where the peptide is Carnosine®, the compositions preferably contain from about 0.1% to about 5%, by weight of the composition, of such peptides. In other embodiments wherein the peptide or peptide-containing composition palmitoyl-lys-thr-thr-lys-ser and/or Biopeptide CL® are included, the compositions preferably contain from about 0.0001% to about 10%, of palmitoyl-lys-thr-thr-lys-ser and/or Biopeptide CL® peptide-containing composition.

Flavonoid compounds may also be useful herein and include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C₁-C₈ alkyl, C₁-C₄ alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof.

In one particularly preferred embodiment of the present invention, the glycoside flavonoid is selected from the group consisting of glucosyl hesperidin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds.

Anti-acne agents useful herein include salicylic acid, 4-methoxysalicylic acid, benzoyl peroxide, lactic acid, metronidazole, panthenol, retinoic acid and its derivatives, sulphur, triclosan, and mixtures thereof.

Anti-oxidants and radical scavengers useful herein include, for example, tocopherol (vitamin E), esters of tocopherol such as tocopherol acetate and tocopherol nicotinate, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, amines (i.e., N,N-diethylhydroxylamine, amino-guanidine), sulfhydryl compounds (i.e., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts.

Anti-inflammatory agents useful herein include, for example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, and the licorice (the plant genus/species Glycyrrhiza glabra) family including glycyrrhetic acid, glycyrrhizic acid, and derivatives thereof (e.g., salts and esters).

Antimicrobial agents useful in the present invention include benzoyl peroxide, erythromycin, tetracycline, clindamycin, azelaic acid, sulfur resorcinol phenoxyethanol, and IRGASAN® DP 300 (Ciba Geigy Corp., U.S.A.).

Skin texture improvement agents useful herein include niacinamide, esters of nicotinic acid, nicotinyl alcohol, panthenol, panthenyl ethyl ether, n-acetyl cysteine, n-acetyl-L-serine, phosphodiesterase inhibitors, trimethyl glycine, tocopheryl nicotinate, and vitamin B₃ and analogues or derivatives, and mixtures thereof. Panthenol is particularly preferred. Panthenol is commercially available, for example, by Roche. Skin vitalizing agents useful herein include seaweed extracts such as algae extract and Laminaria Digitata extract.

In addition, the treatment composition preferably includes one or more aqueous carriers. The level and species of the carrier are selected according to the compatibility with other components, and other desired characteristic of the product. The aqueous carrier is contained in the composition at a level by weight of, preferably from about 30% to about 99%, more preferably from about 50% to about 95%, still more preferably from about 70% to about 95%.

Carriers useful in the present invention include water and water solutions of lower alkyl alcohols. Lower alkyl alcohols useful herein are monohydric alcohols having 1 to 6 carbons, more preferably ethanol. Preferably, the aqueous carrier is substantially water. Deionized water is preferably used. Water from natural sources including mineral cations can also be used, depending on the desired characteristic of the product.

The pH of the treatment composition is preferably from about 6 to about 8. When skin car actives are included in the treatment composition, the pH may be adjusted to that which provides optimum efficacy of the active skin benefit agents. Buffers and other pH adjusting agents can be included to achieve the desirable pH. Suitable pH adjusters herein include acetates, phosphates, citric acid, citrates such as sodium, triethanolamines and carbonates.

The treatment composition may also include preservatives and preservative enhancers such as water-soluble or dispersible preservatives including methyl paraben, ethyl paraben, propyl paraben, imidazolidinyl urea, Germall 115, methyl, ethyl, propyl and butyl esters of hydroxybenzoic acid, benzyl alcohol, EDTA, Bronopol (2-bromo-2-nitropropane-1,3-diol), sodium salicyclate, phenoxypropanol, ethyl alcohol, iso-propyl alcohol, butylene glycol, pentylene glycol, hexylene glycol, and mixtures thereof. Other preservatives and preservative enhancers that that commonly used in cosmetic/skincare and quasi drug products may further be suitable for use herein.

In addition to the above described components, the treatment composition of the present invention may further include; ultraviolet light absorbers or scattering agents; sequestrants; anti-androgens; depilation agents; soluble or colloidally-soluble moisturizing agents such as hyaluronic acid and starch-grafted sodium polyacrylates such as SANWET® IM-1000, IM-1500 and IM-2500 available from Celanese Superabsorbent Materials, Portsmith, Va., USA and described in U.S. Pat. No. 4,076,663; proteins and polypeptides and derivatives thereof; organic hydroxy acids; drug astringents; external analgesics; film formers; anticaking agents; antifoaming agents; binders; coloring agents; perfumes, essential oils, and solubilizers thereof; natural extracts; guai-azulene; and yeast ferment filtrate. Cooling agents such as menthol, camphor, menthol derivatives including menthyl lactate, menthoxy propanediol, menthyl glucoside, and mixtures thereof, may also be provided.

Method of Preparation

In a preferred embodiment, the cosmetic treatment article is a mask composition comprised of a water-insoluble substrate and a gel composition, and that is preferably further comprised of a treatment composition. This embodiment is believed to provide maximum ease of use benefit to the user, because all the user needs to do is remove the article from the packaging and apply it to the skin. In another embodiment, the article is comprised of a water-insoluble substrate and gel composition. In this embodiment, any treatment composition can be separately applied to the skin or to the pre-gellated substrate.

In other preferred embodiments, the article is provided as a kit comprised of a water-insoluble substrate, with one or more of the first gel forming composition, the second gel forming composition, or the treatment composition maintained separate from the substrate and/or from one another, until the time of use. In such an embodiment the user himself or herself may create the gel composition by preparing the treatment article as described herein.

A preferred embodiment of a mask composition of the present invention can be made as follows. Generally, the mask composition is made by first sufficiently coating the water-insoluble substrate with the first gel forming composition. After coating, the coated water-insoluble substrate is provided with the second gel forming composition, for example by spraying, preferably on both sides. This causes the formation of the gel that permeates the water-insoluble substrate. Once the gel has sufficiently viscosified, preferably a treatment composition is uniformly coated or sprayed onto the gel that has permeated the substrate. Without being bound by theory it is believed that the treatment composition does not penetrate into the gel, although some of the water or aqueous carrier present in the treatment composition is likely to enter into the gel structure. Thus, most of the treatment composition is available for contact with the skin.

In another preferred embodiment, after the first and second gel forming compositions have been applied to the substrate as above, instead of uniformly spraying or coating a treatment composition over the entire gel-permeated substrate, selective spraying or coating of the treatment composition can be performed. In this embodiment selected locations of the gel-permeated substrate may be provided with different treatment compositions. For example, a treatment composition containing a high level of skin moisture benefit agent can be provided at the cheek locations of the substrate, while a different treatment composition containing less skin moisture benefit agent and/or an anti-acne benefit agent could be applied to the nose location of the same gel-permeated substrate. Similarly, anti-wrinkle agents could selectively be provided at the eye contour area and/or at the nasolabial fold locations. There are many variations for providing a desired benefit agent to a desired area of the skin by selectively applying a treatment composition to the corresponding location of the substrate, and all are within the scope of the present invention. Such targeting of skin benefit agents was not previously possible with substrate masks that were made by pouring a liquid composition into a housing and soaking a mask substrate in the liquid composition.

When selective, targeted application of treatment compositions is provided, it may further be desirable to provide diffusion barriers to prevent bleeding or undesired mixing of the different treatment compositions that were applied to different areas of the substrate. For example, diffusion barriers could include physical means to cut the diffusion paths, such as air bubbles, plastic barriers, and the like.

Preferably, the cosmetic treatment article or cosmetic treatment article kit is housed in a package(s) that is hermetically sealed and opened upon use. In addition, the present mask composition may comprise an applicator to provide even easier and more hygienic use. Any type of applicator by which extraneous contact with the hands of the user during application of the article is avoided may be provided. For example, a release liner may be provided on one side of the substrate; when the mask is to be used, the liner is peeled off and the mask applied to the skin. When a release liner is used, the mask composition may rolled-up and placed inside a hermetically sealed pouch.

Method of Use

The mask composition of the present invention is suitable for topical application on human body skin, particularly facial skin. The use of the present composition provides skin conditioning benefits such as smoothness, softness, and moisturized feel to the skin due to the deposition and penetration of skin benefit agents. Other benefits to the skin can be provided by application of the present mask composition in view of the specific benefit agents included in the treatment composition. The mask composition of the present invention is particularly advantageous in delivering the humectant and other benefit agents in that the skin is exposed to an abundant amount of such agents over a lengthy period of time. Compared to when a liquid composition is applied to the skin without the use of the insoluble substrate, the mask composition of the present invention with the gel composition as a delivery means is believed to provide better distribution and deposition of such agents, and better penetration of those agents which are percutaneously deliverable. Further, when an insoluble substrate having low air permeability is used, more effective penetration of the skin benefit agents into the skin is expected. Compared to when a liquid saturated mask composition is used, the mask composition of the present invention provides a different use experience and a more convenient application experience. The mask composition of the present invention is also believed to provide emotional benefits to the user upon use, such as refreshing feel, and relaxation feel.

In one preferred embodiment, the mask composition is used to treat the facial skin by the steps of:
(a) applying the mask composition to the majority of the area of the facial skin;
(b) allowing the mask composition to stand on the facial skin for a period of time no longer than until any portion of the mask composition is dried;
(c) removing the mask composition from the facial skin; and
(d) optionally, massaging the remainder treatment composition left on the facial skin into the skin.

The mask fits to the facial skin by gently placing on the skin. For better fit the mask is pressed to the facial skin using finger tips.

As used herein, “dried” refers to a state wherein water and other volatile components such as perfume, if included, evaporates from the water insoluble substrate, thereby leaving the substrate significantly less capable of delivering the benefit agents to the skin. Thus, once a portion of the mask is dried, even distribution of the benefit agents cannot be expected. Further, when dried, the mask composition provides an unpleasant stiff and tough feeling to the skin when applied.

Because the mask composition of the present invention is easily dried via exposure to regular atmospheric conditions, the mask composition must be housed in a hermetically sealed package during storage.

The period of time required until dried portions appear will depend on the atmosphere in which the use takes place, i.e. temperature, humidity, air circulation; and the structure and body temperature of the user. Typically, the mask composition should be designed so that no dried portions appear within a period of about 15 minutes when used in room temperature at a humidity of about 50%. When an insoluble substrate having low air permeability is used, the period of time by which the mask composition is dried can be prolonged, preferably from about 5 to about 45 minutes.

In another preferred embodiment, a mask composition kit is used to treat the facial skin by the steps of:
(a) applying the first gel forming composition to the substrate;
(b) applying the second gel forming composition to the substrate;
(c) applying one or more treatment composition(s) to the substrate, or to selected locations of the substrate;
(d) applying the mask composition to the majority of the area of the facial skin;
(e) allowing the mask composition to stand on the facial skin for a period of time no longer than until any portion of the mask composition is dried;
(f) removing the mask composition from the facial skin; and
(g) optionally, massaging the remainder treatment composition left on the facial skin into the skin.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

First Gel Forming Composition

| Component | Ex. 1 | Ex. 2 | Ex. 3 | Ex. 4 | Ex. 5 | Ex. 6 |
|---|---|---|---|---|---|---|
| Potassium Alginate *1 | 1.0 | — | — | 1.0 | — | — |
| Sodium Alginate *2 | — | 1.0 | — | — | 1.0 | — |
| Sodium Pectate | — | — | 1.0 | — | — | 1.0 |
| Sodium Salicylate | 1.0 | 1.0 | 1.0 | 1.0 | 1.0 | 1.0 |
| Methylparaben | 0.1 | 0.1 | 0.1 | 0.1 | 0.1 | 0.1 |
| Benzyl Alcohol | 0.15 | 0.15 | 0.15 | 0.15 | 0.15 | 0.15 |
| Glycerin | 10.0 | 10.0 | 10.0 | — | — | — |
| 1,3-Butylene Glycol | — | — | — | 10.0 | 10.0 | 10.0 |

| | |
|---|---|
| Deionized Water | q.s. to 100% |

Definitions of Components
*1 Potassium Alginate: Kimica Algin K available from Kimika
corporation
*2 Sodium Alginate: Kimica Algin Na available from Kimika corporation

Second Gel Forming Composition

| Component | Ex. 1 | Ex. 2 | Ex. 3 | Ex. 4 |
|---|---|---|---|---|
| Calcium Chloride | 1.0 | — | — | — |
| Aluminum Chloride | — | 0.5 | — | — |
| Calcium Citrate | — | — | 1.0 | — |
| Ferric Chloride | — | — | — | 1.0 |
| Methylparaben | 0.1 | 0.1 | 0.1 | 0.1 |
| Benzyl Alcohol | 0.15 | 0.15 | 0.15 | 0.15 |

| | |
|---|---|
| Deionized Water | q.s. to 100% |

Treatment Composition

| Component | Ex. 1 | Ex. 2 | Ex. 3 | Ex. 4 | Ex. 5 | Ex. 6 |
|---|---|---|---|---|---|---|
| Laponite *1 | 4 | 4 | 1 | 2 | 3 | 5 |
| Bentonite *2 | — | — | 5 | — | — | — |
| Glycerin | — | 5 | 2 | — | 5 | 2 |
| Butylene Glycol | 10 | — | 2 | 2 | 5 | 10 |
| Dipropylene Glycol | — | 5 | — | 2 | — | 2 |
| Menthyl Lactate | 0.015 | 0.015 | 0.015 | 0.015 | 0.015 | 0.015 |
| Benzyl Alcohol | 0.15 | 0.15 | 0.15 | 0.15 | 0.15 | 0.15 |
| Disodium EDTA | 0.11 | 0.11 | 0.11 | 0.11 | 0.11 | 0.11 |
| Methylparaben | 0.1 | 0.1 | 0.1 | 0.1 | 0.1 | 0.1 |
| Citric Acid | 0.092 | 0.1 | 0.092 | 0.092 | 0.092 | 0.1 |
| Sodium Citrate | 0.025 | 0.01 | 0.025 | 0.025 | 0.025 | 0.01 |
| Niacinamide *3 | — | 3.5 | — | — | — | 2.0 |
| Panthenol *4 | 1.0 | — | 1.0 | 1.0 | — | — |
| Mulberry Extract *5 | 0.5 | — | — | — | — | — |
| Natural Extracts *6 | — | 0.05 | — | — | — | — |
| Green Tea in | — | — | — | — | — | 1.0 |
| Glycerin with Water *7 | | | | | | |
| Palmitoyl Lys Thr | — | — | — | — | — | 0.4 |
| Thr Lys Ser *8 | | | | | | |
| Perfume | — | 0.015 | — | 0.015 | — | 0.015 |

| | |
|---|---|
| Deionized Water | q.s. to 100% |

Definitions of Components
*1 Laponite: XLG available from Rockwood Additives, Ltd.
*2 Bentonite: Bentolite H from Southern Clay Products
*3 Niacinamide: Niacinamide available from Roche
*4 Panthenol: DL-Panthenol available from Roche
*5 Mulberry Extract: Mulberry Extract BG available from Maruzen
Pharmaceuticals
*6 Natural Extracts: Phytelene EGX 243 [A mixture of Arnica Mountan
Flower Extract, Hypericum Perforatum Extract, Ivy (Hedera Helix)
extract, witchhazel (Hamamelis Virginiana) extract, grape
(vitis vinifera) Leaf extract, and horse chestnut (aesculus hippocastanum)
extract] available from Kaneda.
*7 Green Tea in Glycerin with Water: available from Bio-Botanica,
Hauppauge, NY
*8 Palmitoyl Lys Thr Thr Lys Ser: Peptide available from Sederma

The mask compositions of Examples 1 through 6 are made of about 0.7 g of substrate Havix 2365, cut and shaped according to FIG. 1, coated with about 20.0-25.0 g of a first gel forming composition, then coated with about 20.0-25.0 g of a second gel forming composition, followed by removal of excess water from surface of the gel that forms. Then, preferably either uniformly or selectively coat with about 4.0 g of a treatment composition or compositions. The mask compositions can also be made of about 1.8 g of Kuraray TT463Q60 substrate or about 3.5 g of cotton substrate, instead of the Havix substrate specified above.

Method of Preparation

The compositions above described as can be made by any method known to the artisan. The compositions are suitably made as follows:

First Gel Forming Composition:

1. All components except for water soluble polymeric gelling agent are dissolved in water and mixed until homogeneous. Heat may be added as necessary.

2. The water soluble polymeric gelling agent is added to the products of step (1) and mixed until homogeneous, using a high speed mixer as necessary.

Second Gel Forming Composition:

1. All components are dissolved in water and mixed until homogeneous.

Treatment Composition:

1. Rheology modifier is pre-dispersed into water soluble humectant in a vessel, if present. If included, other remaining components such as perfumes are added to this vessel.

2. pH adjusting agent is dissolved in water in another vessel, and mixed until homogeneous

3. All components are dissolved in water and mixed until homogeneous in another vessel.

4. The products of step (1) and step (3) are mixed until homogeneous, using a high speed mixer as necessary.

5. The products of step (2) is added to the product of step (4) when product viscosity of step (4) become high.

6. The produce of step (5) is mixed until homogeneous, using a high speed mixer as necessary.

The embodiments of the present invention disclosed and represented above have many advantages. When applied to the face using finger tips for good fit and left for about 15 minutes, they provide improved skin conditioning benefits such as smoothness, softness, and moisturized feel to the skin.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. What is claimed is:

1. A cosmetic treatment article comprising:
(1) a water insoluble substrate; and
(2) a gel composition comprising:
(a) a first gel forming composition comprising a water soluble polymeric gelling agent;
(b) a second gel forming composition comprising a gel strengthening agent; and
(3) a treatment composition comprising a rheology modifier.

2. The cosmetic treatment article of claim 1 wherein the water soluble polymeric gelling agent is selected from the group consisting of synthetic polymers, natural polymers, and mixtures thereof.

3. The cosmetic treatment article of claim 2 wherein the natural polymers are selected from the group consisting of gelatin, polysaccharides, and mixtures thereof.

4. The cosmetic treatment article of claim 3 wherein the polysaccharide is selected from the group consisting of red seaweed polysaccharides, glucomannans, galactomannans, fermentation polysaccharides or derivatives thereof, brown seaweed polysaccarides, extracts of marine invertebrates, starch or derivatives thereof, natural fruit extracts, plant fiber derivatives, kelp, natural plant exudates and resinous gums, and mixtures thereof.

5. The cosmetic treatment article of claim 4 wherein the red seaweed polysaccharide is an alginate.

6. The cosmetic treatment article of claim 1 wherein the gel strengthening agent is selected from the group consisting of sugar, alcohol, any monovalent salt or multivalent salt or metal ion, and mixtures thereof.

7. The cosmetic treatment article of claim 6 wherein the gel strengthening agent is a multivalent metal ion.

8. The cosmetic treatment article of claim 1 wherein the rheology modifier is selected from the group consisting of synthetic thickeners, natural clays, and mixtures thereof.

9. The cosmetic treatment article of claim 8 wherein the rheology modifier is laponite.

10. The cosmetic treatment article of claim 1 wherein the treatment composition further comprises at least one skin benefit agent.

11. The cosmetic treatment article of claim 10 wherein the skin benefit agent is selected from the group consisting of water soluble humectants, chronic whitening agents, skin tone changing agents, peptides, flavonoids, anti-oxidants, cleansing agents, free radical scavengers, moisturizers, anti-acne agents, anti-dandruff agents, anti-aging agents, softeners, anti-wrinkle agents, keratolic agents, anti-inflamatory agents, skin texture treatment agents, fresheners, healing agents, liporegulators, vascular protectors, anti-bacterials, agents, anti-fungal agents, anti-perspirant agents, deodorants, skin conditioners, anesthetics, nourishing agents, sebum absorbers, and moisture absorbers, and mixtures thereof.

12. The cosmetic treatment article according to claim 1, wherein the article is a mask composition configured to cover the majority of the area of human facial skin.

13. The cosmetic treatment article of claim 1 wherein the water-insoluble substrate has a thickness of from about 100 μm to about 1 cm.

14. The mask composition of claim 1 wherein the water-insoluble substrate is at least partially made of hydrophilic materials selected from cotton, pulp, rayon, and mixtures thereof.

15. A method of treating the facial skin comprising the steps of:
(a) applying to the majority of the area of the facial skin a cosmetic treatment article comprising:
i) a water insoluble substrate; and
ii) a gel composition comprising a first gel forming composition comprising a water soluble polymeric gelling agent, and a second gel forming composition comprising a gel strengthening agent; and
iii) a treatment composition comprising a rheology modifier;
(b) allowing the mask composition to stand on the facial skin for a period of time no longer than until any portion of the mask composition is dried; and
(c) removing the mask composition from the facial skin.

16. The method according to claim 15, further comprising the step of massaging into the skin the remaining treatment composition left on the facial skin after applying the cosmetic treatment article.

17. The method of any of claim 15 wherein the period of time is from about 5 to about 45 minutes.

18. A cosmetic treatment article comprising:
(1) a water insoluble substrate; and
(2) a gel composition comprising:
(a) a first gel forming composition comprising a water soluble polymeric gelling agent;
(b) a second gel forming composition comprising a gel strengthening agent; and
(3) at least two different treatment compositions, wherein at least one of the treatment compositions comprises a rheology modifier, and wherein each treatment composition is provided at one or more selected locations on the water insoluble substrate.

19. A cosmetic treatment article kit comprising:
(1) a water insoluble substrate; and
(2) a gel composition comprising:
(c) a first gel forming composition comprising a water soluble polymeric gelling agent;
(d) a second gel forming composition comprising a gel strengthening agent; and
(3) a treatment composition comprising a rheology modifier;
wherein the water insoluble substrate is maintained separate from at least one of the first gel forming composition, the second gel forming composition, and the treatment composition until the time of use of the article.
